(19) United States
(12) Patent Application Publication
(10) Pub. No.: US 2021/0219636 A1
(43) Pub. Date: Jul. 22, 2021

(54) ULTRAVIOLET FACE MASK

(71) Applicants: Alix Stroiazzo-Mougin, Boca Raton, FL (US); Carolina Maldonado, Boca Raton, FL (US)

(72) Inventors: Alix Stroiazzo-Mougin, Boca Raton, FL (US); Carolina Maldonado, Boca Raton, FL (US)

(21) Appl. No.: 17/222,738

(22) Filed: Apr. 5, 2021

Related U.S. Application Data
(60) Provisional application No. 63/005,192, filed on Apr. 3, 2020.

(51) Int. Cl. A41D 13/11; A62B 23/02; A61L 2/10

(52) U.S. Cl. CPC A41D 13/1184; A61L 2/10; A41D 13/1192; A62B 23/02

(57) ABSTRACT

A face mask assembly that having a transparent cover for covering the nose and mouth of a wearer, an air ventilation unit for purifying and sterilizing the inhaled and exhaled air, and a liner coupled along a periphery of the transparent cover. The air ventilation unit includes a filter media for filtering the air to be inhaled and one or more UV-C LEDs to irradiate the filter media and an inner volume of the air ventilation unit.

### CROSS-REFERENCE TO RELATED APPLICATIONS

This application claims priority to the U.S. provisional patent application Ser. No. 63/005,192, filed on Apr. 3, 2020, which is incorporated herein by reference in its entirety.

### FIELD OF INVENTION

The application relates to a face mask assembly, and more particularly, this application relates to a face mask assembly having a transparent body and UV-C disinfection module.

### BACKGROUND

Face masks covering the nose and mouth are widely used as a shield against airborne pathogenic microorganisms. A typical facemask is having a porous structure that filters the particulate material including the microbes from the breathing air. What can be filtered by a face mask depends upon its pore size. Accordingly, a range of face masks is commercially available having different pore sizes for different applications. Bedsides the filtration, a facemask may also have a layer of charged fabric layer that can capture minute particles, for example, N95 face masks have a layer of electrocharged material that attracts virions.

Although face masks are available that can protect against a range of microorganisms. However, the known face masks suffer from two major drawbacks. First, the facemask hides almost half of the face, and in particular the lips of the wearer of the facemask. Hiding the face by the face mask affects social interactions. People with hearing disorders who rely on lip-reading also faces issue in communicating with people wearing face masks. Second, the retained microorganisms on the face mask get accumulated in the layers of the face mask. This not only decreases the efficiency of the face mask but also the face mask can itself become a source of contamination. Frequency replacing of the face mask poses an additional economic burden. Wearing a face mask is important during pandemics, such as the Covid-19 pandemic, to decrease the spread of the virus.

Thus, an urgent need is there for a face mask that is devoid of the aforesaid drawbacks of the conventional face masks.

### SUMMARY OF THE INVENTION

The principal object of the present invention is therefore directed to a face mask assembly that does not hide the lips of a person wearing the face mask.

It is another object of the present invention that the face mask assembly provides additional safety by directly killing the microorganisms.

It is still another object of the present invention that the face mask assembly has a long useful life making it economical in use.

It is still a further object of the present invention that the face mask assembly can be customized by replacing the filter.

It is still another object of the present invention that the face mask assembly prevents the deposition of microbes in the filter material.

It is still an additional object of the present invention that the facemask assembly provides for sterilization of both inhaled and exhaled air.

In one aspect, disclosed is a face mask assembly having a transparent cover dimensioned to cover the nose and mouth of a wearer. A lining of the soft material, such as silicon around the periphery of the cover, the lining having contours that resemble the contours of a face, such as a face mask assembly can snugly fit onto the face around the nose and mount, substantially forming an airtight seal. An air ventilation unit can be configured near the bottom of the face makes assembly, the air ventilation unit is configured to provide an exchange of air between an inner volume of the face mask assembly worn by a wearer and atmosphere. The air ventilation unit includes one or more vents through which air can ingress into the air ventilation unit or egress from the air ventilation unit. An inner volume of the air filtration unit and the inner volume of the face mask assembly in fluid communication through a filter medium, wherein breathing air from the inner volume of the air filtration unit is filtered through the filter medium and the filtered air enters the inner volume of the facemask assembly for breathing. The air ventilation unit also includes a disinfecting radiation source, such as Far UV-C LEDs that irradiate the filter media and the inner volume of the air filtration unit.

In one aspect, the transparent cover can have a fog inhibit layer that prevents the formation of fog on the inner and outer surfaces of the transparent cover.

In one aspect, the radiation source can be powered by a rechargeable battery and a port, such as a USB port can be provided for charging the rechargeable battery. In one case, the port can be provided adjacent the one or more vents in the base of the air ventilation unit.

In one case, the air ventilation unit includes a filter lid and filter shelf that sandwiches the filter media. The filter lid and filter shelf mounted in a housing of the air ventilation unit, such as they can be removed to replace the filter media. The filter lid and filter shelf can be accessed through the face opening of the cover.

can be removed to replace the filter media. The filter media can be any filter media, such as MERV16 and HEPA filters.

In one case, the face mask assembly includes a pair of straps, each having a proximal end and distal end, the proximal end removably coupled to the cover member, and the distal end having an ear loop that can be worn around the ears. The straps having a length adjusting mechanism for adjusting the length of the straps according to the size of a face.

These and other objects and advantages of the embodiments herein will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a perspective view of a face mask assembly, according to an exemplary embodiment of the present invention.

FIG. 2 is a bottom view of the face mask assembly having the vents for air, according to an exemplary embodiment of the present invention.

FIG. 3 is an exploded view of the face mask assembly showing a filter lid, a filter media, and a radiation source module, according to an exemplary embodiment of the present invention.

FIG. 4 shows a housing of an air ventilation unit of the facemask assembly having the radiation source module, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

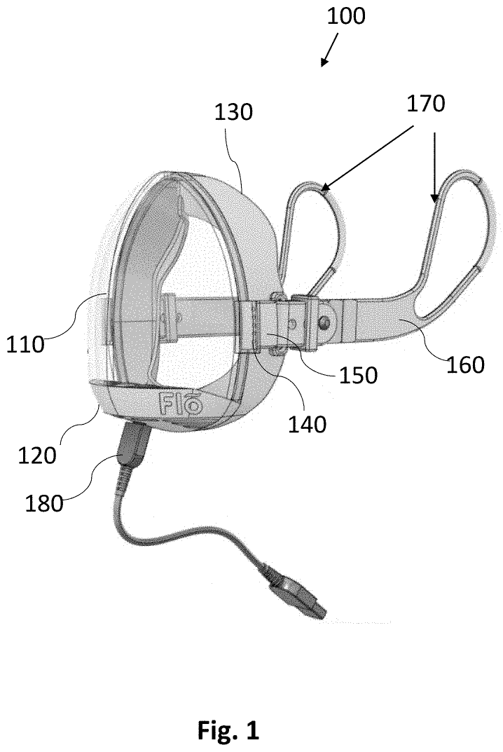

Referring to FIG. 1, which is a perspective view of the disclosed face mask assembly 100. The face mask assembly 100 can include a transparent cover 110 that can cover the mouth and nose of a face. The top of cover 110 can be narrow to cover the nose and the bottom of the cover can be broad for covering the mouth. The transparent cover 110 can be rigid and does not touch either the nose or mouth. Sufficient space may exist between the skin of the wearer and the transparent cover 110 to avoid any discomfort. The transparent cover can be made of any lightweight and transparent material, such as plastics. The transparent cover can be impervious to air. The face of the wearer can be visible from the transparent cover 110. The material of the cover can be skin-friendly and safe for use in face masks. The transparent cover 110 can substantially resemble a conical shape having a flat bottom. The transparent cover 100 can have suitable coatings to prevent the buildup of fog on the surface of the transparent cover 100. Additionally, other coatings, such as for preventing scratches on the transparent cover, UV protective coatings, and for preventing smudges on the transparent cover can also be provided.

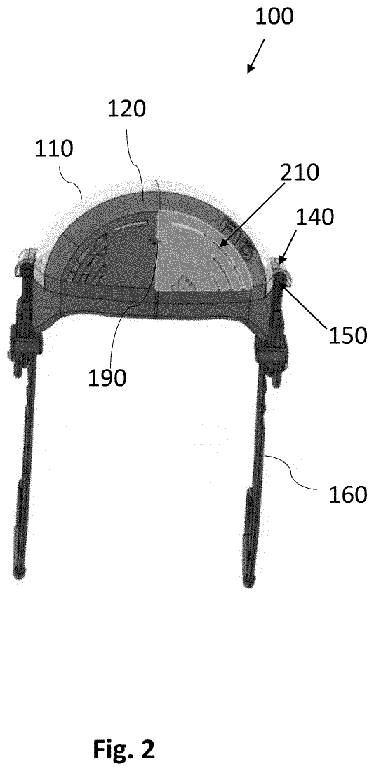

The disclosed face mask assembly may also include an air ventilation unit 120 at the bottom. The air ventilation unit 120 can include a housing that can be coupled to the bottom of the face mask cover 110. The housing of the air ventilation unit 120 can be fixedly or removably coupled to the face mask cover 110. The housing of the air ventilation unit 120 can be transparent or opaque and can have vents for the air. FIG. 2 shows the bottom view of the face mask assembly 100. Air vents 210 can be seen at bottom of the housing. Through these vents, air can ingress or egress from the air ventilation unit 120. The shape and distribution of the air vents can be varied, without departing from the scope of the present invention. The air ventilation unit 120 can provide for filtration and sterilization of breathing air.

Referring to FIG. 1, the face mask assembly 100 may also include a lining 130 of soft material along the periphery of the cover 110. The lining comes in contact with the face of a person wearing the disclosed face mask assembly. The lining 130 can have contours that can match the contours of a face around the nose and mouth. The soft material of the lining 130 the contours allow the face mask assembly to fit over a face of a wearer. Preferably, an airtight seal can be formed by the lining 110 on the face of the person to prevent ingress of air through gaps between the lining and the skin. The softness of the material may prevent any discomfort to the wearer. The soft material of the lining 130 can be any skin-friendly material, such as silicon. The lining 130 can be manufactured in a range of colors for aesthetic appearance. In case, cover 110 is having a continuous periphery along with the face opening, continuous lining 130 can be coupled with cover 110. In case, the cover member 110 and the housing of the air ventilation unit 120 form the face opening, the continuous silicon lining can be coupled to face cover 110 and portion of the housing of the air ventilation unit 120.

The cover 110 can also have a pair of brackets 140 on its opposite sides. These brackets 140 can be used to coupled straps for wearing the facemask assembly. A loop member 150 can be seen slide fit into the brackets. The other end of the loop member can have a loop to which strap 160 can be coupled. The straps can run around the head of the wearer for supporting the face mask assembly 100. Alternately, the two straps 160 shown in FIG. 1, can have ear loops 170 that can be worn around the ear. The length of the strap 160 can be adjusted, for example, FIG. 1 shows a belt and buckle mechanism for adjusting the length of the straps. The straps can be made of any skin-friendly material that is also comfortable to the wearer. In one case, the straps can also be made of silicon.

FIG. 1 also shows a charging cable extending from the bottom of the air ventilation unit 120 for illustration purposes only. The charging port (190 shown in FIG. 2) can be provided to which the charging cable can be plugged.

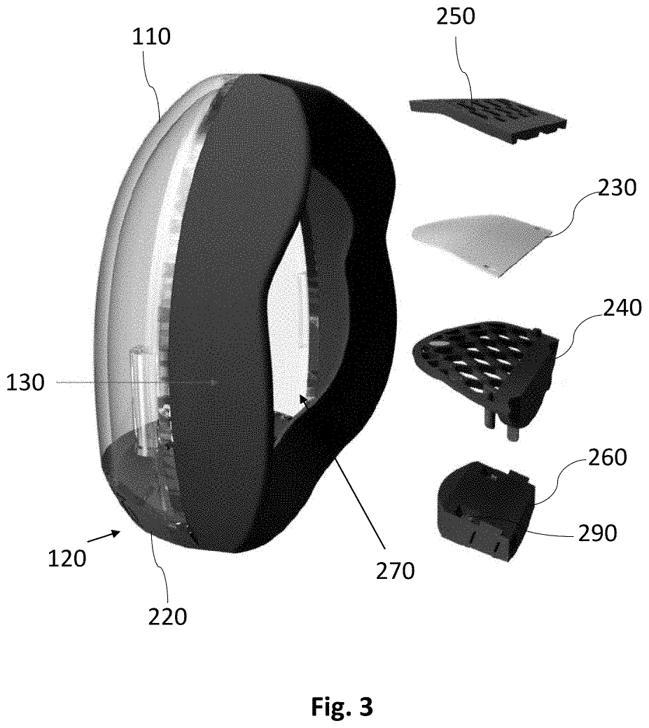

Referring to FIG. 3, which shows an exploded view of the face mask assembly 100 having transparent cover 110, housing 220, and the silicon lining 130 fixedly coupled to form a unit. The air ventilation unit 120 can include the housing 220 which encloses a filter media 230 sandwiched between a filter lid 250 and a filter shelf 240. The air ventilation unit 120 can also include a radiation module 260 that can enclose a PCB having the radiation source, for example, a printed circuit board having a pair of UV-C LEDs.

The filter media 230 can be any filter media known to be used for filtration of breathing air. For example, filter media such as MERV16 and HEPA filters are within the scope of the present invention. A user can replace the filter media with any other type of filter media without departing from the scope of the present invention. For example, the user can use either MERV16 or HEPA filters. Such filter media can be provided separately. The filter media 230 can be supported and protected by the filter lid 250 on top and the filter shelf 240 at the bottom. The filter lid 250 can removably lock to the filter shelf 240 securing the sandwiched filter media 230. Both the filter lid 250 and the filter shelf 240 can be of a honeycomb structure of grill for supporting the filter media and provided a free passage to the air. The filter lid 250 and the filter shelf 240 can mount in the housing 220 forming an airtight seal, such as the air can only pass through the filter media. The filter media can act as a barrier between an inner volume of the face mask assembly, the inner volume between the cover 110 and the face of the wearer, and an inner volume of the air ventilation unit 120.

The air can ingress into the inner volume of the air ventilation unit 120 through the vents in base 210 of the housing 220. During inhalation, the air passes through the filter media into the inner volume of the face mask assembly. While passing, the air can be filtered. The filter shelf 240 can be snap-fit onto the radiation module 260, such as a user can reach the filter lid 250 coupled to the filter shelf 240 through the face opening 270 and pull out the filter lid 250 coupled to the filter shelf 240. The filter lid 250 can then be separated to gain access to the filter media. The filter media 230n can be cleaned or replaced. The cleaned or replaced filter media can be placed on the filter shelf 240 and secured by the filter lid 250. Thereafter, the filter shelf 240 can be mounted to the radiation module 260. Alternatively, the filter shelf 240 can also be mounted to the housing 220 independent of the radiation module 260. A release mechanism, such as a spring urged release mechanism can also be provided for releasing the filter shelf 240 from the housing, which can then be removed from the face opening 270 of the face mask assembly.

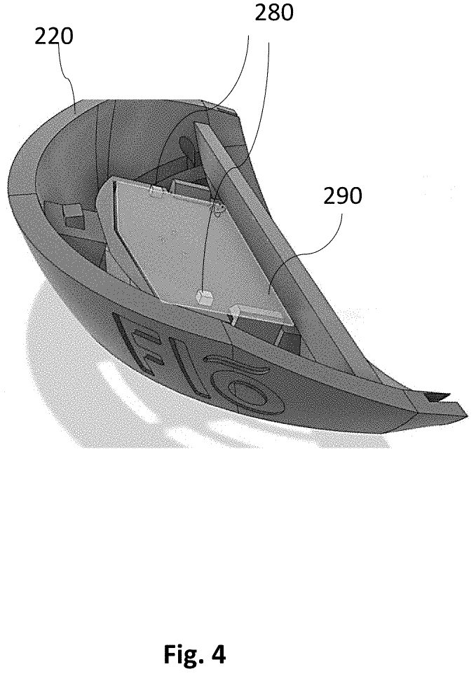

The radiation module 260 can be coupled to the housing 220, for example, through fasteners. In one case, the radiation module 260 can be coupled to the base of the housing 220 adjacent to the air vents. Air drawn from the vents can pass through the surrounding of the radiation module 260 towards the filter media. The radiation module 260 can enclose a chipset having a radiation source, such as UV-C LEDs. FIG. 4 shows the radiation module 260 without its top cover showing a printed circuit board 290 having a pair of UV-C LEDs 280. The pair of UV-C LEDs faces upward in direction of the filter media, such as to irradiate the filter media mounted over the radiation module. Additionally, the inner volume of the air ventilation unit can be irradiated by UV-C light. This prevents the deposition of microbes in layers of the filter media prolonging the life and enhancing the safety of the filter media. Moreover, both the air to be inhaled and the exhaled air can be sterilized in the air ventilation unit. The UV-C LEDs and the chipset can be powered by a rechargeable battery (not shown) also enclosed in the radiation module 260. The rechargeable battery can be a lithium-ion battery, or any other rechargeable battery known to a skilled person and any such rechargeable battery is within the scope of the present invention. Additionally, the radiation module can be dissembled to replace a dead battery. A charging circuitry can also be provided to charge the battery. Such charging circuitry can be connected to a charging port 190 provided in the housing 220. Additionally, LED indicators, such as battery status, charging status, and like can also be provided in housing 220. The charging circuitry may also trigger audio alerts, such as beeps to indicate a dying battery.

An on/off switch can also be provided in housing 220 that may allow turning the UV-C LEDs on and off. Alternatively, sensors can be embodied in the assembly that can sense the use of the facemask assembly i.e., when a person wears the face mask assembly and then remove the same. The sensors can automatically trigger the turning on and off the UV-C LEDs based on the status of the facemask assembly.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A face mask assembly comprising:
a transparent cover dimensioned to cover a nose and mouth of a wearer without coming in contact with skin of the wearer; the transparent cover having a bottom and a periphery;
a liner coupled to the face opening and running along its length forms a face opening, the liner having contours corresponding to contours of a face around the nose and mouth of the wearer; and
an air ventilation unit coupled to the bottom of the transparent cover, wherein the air ventilation unit, the transparent cover, and the lining forms an inner volume of the face mask assembly when worn by the wearer, the air ventilation unit comprises:
a filter media configured to form a porous barrier, such as air can be ingress into the inner volume of the face mask assembly from the air ventilation unit only through the filter media, and
UV-C light source configured to irradiate the filter media.

2. The face mask assembly according to claim 1, wherein the air ventilation unit further comprises:
a housing coupled to the transparent cover, the housing having vents for ingress and egress of air between the inner volume of the air ventilation unit and atmosphere;
a radiation module mounted in the housing, the radiation module encloses the UV-C light source;
a filter shelf for mounting the filter media; and
a filter lid configured to secure the filter media on the filter shelf, wherein the filter shelf is removable mounted in the housing.
